(19) United States
(12) Patent Application Publication
(10) Pub. No.: US 2025/0152133 A1
(43) Pub. Date: May 15, 2025

(54) ULTRASOUND DIAGNOSTIC SUPPORT DEVICE, ULTRASOUND DIAGNOSTIC DEVICE, ULTRASOUND DIAGNOSTIC SUPPORT METHOD, AND COMPUTER-READABLE NON-TRANSITORY RECORDING MEDIUM STORING PROGRAM

(71) Applicant: Konica Minolta, Inc., Tokyo (JP)

(72) Inventor: Kazuya TAKAGI, Tokyo (JP)

(21) Appl. No.: 18/941,623

(22) Filed: Nov. 8, 2024

(30) Foreign Application Priority Data
Nov. 14, 2023 (JP) 2023-193672

(51) Int. Cl. A61B 8/00

(52) U.S. Cl. CPC A61B 8/4254; A61B 8/463; A61B 8/465; A61B 8/587

(57) ABSTRACT

The ultrasound diagnostic support device, including: an ultrasound image generator that generates an ultrasound image based on an ultrasound signal from an ultrasound probe that transmits and receives ultrasound waves; a camera image generator that generates a camera image based on an imaging signal from a camera attached to the ultrasound probe; and a diagnostic information manager that stores camera attachment information indicating an attachment state of the camera with respect to the ultrasound probe in association with the ultrasound image and the camera image.

### CROSS REFERENCE TO RELATED APPLICATIONS

The entire disclosure of Japanese Patent Application No. 2023-193672 filed on Nov. 14, 2023 is incorporated herein by reference in its entirety.

### BACKGROUND

### Technological Field

The present invention relates to an ultrasound diagnostic support device, an ultrasound diagnostic device, an ultrasound diagnostic support method, and a program.

### Description of Related Art

In the related art, as a medical image diagnostic device, an ultrasound diagnostic device is known that transmits ultrasound waves toward a subject, receives the reflected waves, and performs predetermined signal processes on the received signals, thereby visualizing the shape, property, or dynamic state of the inside of the subject as an ultrasound image. The ultrasound diagnostic device is safe and imposes a small burden on the subject because the ultrasound diagnostic device can obtain an ultrasound image with a simple operation of applying the ultrasound probe to the body surface or inserting the ultrasound probe into the body.

The ultrasound probe is configured independently of the diagnostic device body and is communicably connected to the diagnostic device body in a wired or wireless manner. The user can freely scan a diagnostic target site by operating the ultrasound probe. On the other hand, due to the high degree of freedom, when the ultrasound image is referred to after diagnosis or shared with others, the probe scanning position is hard to understand, and it is difficult to ensure the reproducibility of the ultrasound image. In order to cope with such a drawback, an ultrasound diagnostic device has been proposed in which the vicinity of an ultrasound probe, specifically, a contact portion between a subject and the ultrasound probe is imaged by a camera, and an ultrasound image and a camera image are managed in association with each other (for example, see Japanese Unexamined Patent Publication No. 2007-282792).

However, in the ultrasound diagnostic device described in PTL 1, the camera image obtained by the probe viewpoint camera has a narrow imaging range and is low in bird's eye view quality. Therefore, the probe scanning position cannot be specified from the camera image, and the diagnostic target site rendered in the ultrasound image may not be accurately grasped. For example, when a landmark (for example, a boundary between a subject and a background or a feature of a body surface) capable of specifying a probe scanning position is not included in the imaging range of the probe viewpoint camera, it is difficult to grasp the probe scanning position from the camera image.

In addition, PTL 1 describes that a probe viewpoint camera and a panoramic camera are used in combination to specify a probe scanning position. However, since a panoramic camera is required, the cost increases. In addition, it is necessary to prevent an obstacle from existing between the probe scanning position and the panoramic camera, which causes a problem in terms of convenience.

### SUMMARY

An object of the present disclosure is to provide an ultrasound diagnostic support device, an ultrasound diagnostic device, an ultrasound diagnostic support method, and a computer-readable non-transitory recording medium storing a program that can easily specify a probe scanning position with respect to a subject and improve the objectivity and reproducibility of ultrasound diagnostic.

### SUMMARY

In order to achieve at least one of the aforementioned objects an ultrasound diagnostic support device reflecting one aspect of the present invention includes: an ultrasound image generator that generates an ultrasound image based on an ultrasound signal from an ultrasound probe that transmits and receives ultrasound waves; a camera image generator that generates a camera image based on an imaging signal from a camera attached to the ultrasound probe; and a diagnostic information manager that stores camera attachment information indicating an attachment state of the camera with respect to the ultrasound probe in association with the ultrasound image and the camera image.

An ultrasound diagnostic device reflecting one aspect of the present invention includes: an ultrasound diagnostic device body to which the ultrasound diagnostic support device is applied; and the ultrasound probe; and the camera.

An ultrasound diagnostic support method reflecting one aspect of the present invention includes: generating an ultrasound image based on an ultrasound signal from an ultrasound probe that transmits and receives ultrasound waves; generating a camera image based on an imaging signal from a camera attached to the ultrasound probe; and storing camera attachment information indicating an attachment state of the camera with respect to the ultrasound probe in association with the ultrasound image and the camera image.

A computer-readable non-transitory recording medium reflecting one aspect of the present invention stores a program for causing a computer to execute predetermined processing, the predetermined processing including: processing of generating an ultrasound image based on an ultrasound signal from an ultrasound probe that transmits and receives ultrasound waves; processing of generating a camera image based on an imaging signal from a camera attached to the ultrasound probe; and processing of storing camera attachment information indicating an attachment state of the camera with respect to the ultrasound probe in association with the ultrasound image and the camera image.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating an appearance of an ultrasound diagnostic device according to an embodiment;

FIGS. 2A and 2B are diagrams illustrating an example of an attachment state of a camera with respect to an ultrasound probe;

FIG. 3 is a block diagram illustrating a main part of a control system of the ultrasound diagnostic device;

FIGS. 4A and 4B are diagrams illustrating an example of a diagnostic screen;

FIG. 5 is a flowchart illustrating an example of an ultrasound diagnostic support processing that is executed when manually setting the camera attachment information;

FIGS. 6A and 6B are diagrams illustrating an example of a setting screen;

FIG. 7 is a flowchart illustrating another example of the ultrasound diagnostic support processing executed in a case where the camera attachment information is manually set;

FIG. 8A to FIG. 8C are diagrams illustrating an example of a setting screen;

FIG. 9 is a flowchart illustrating another example of the ultrasound diagnostic support processing executed when automatically setting camera attachment information; and

FIG. 10 is a diagram illustrating an example of a calibration sheet.

### DETAILED DESCRIPTION OF EMBODIMENTS

Hereinafter, one or more embodiments of the present invention will be described with reference to the drawings. However, the scope of the invention is not limited to the disclosed embodiments.

Hereinafter, embodiments of the present invention will be described in detail with reference to the drawings.

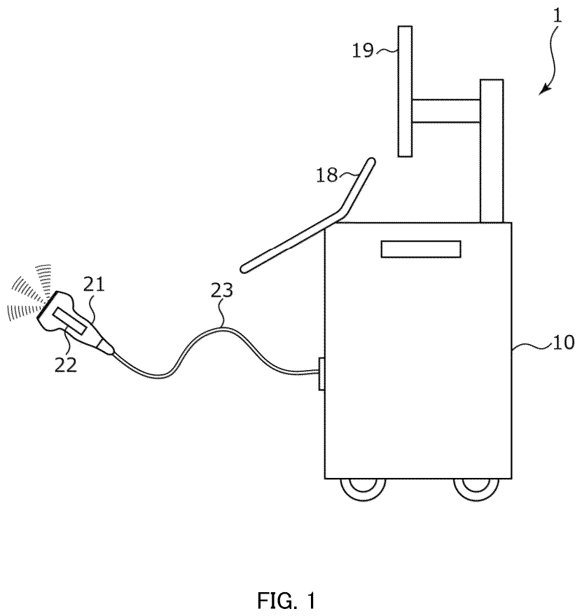

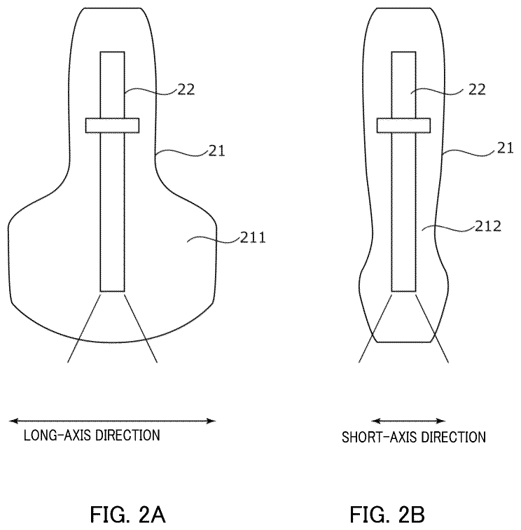

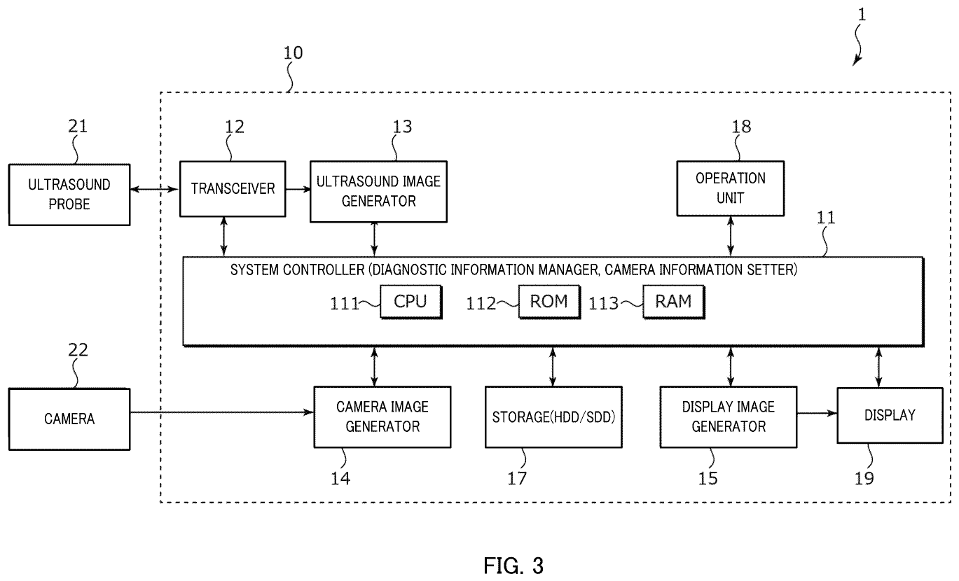

FIG. 1 is a diagram illustrating an appearance of an ultrasound diagnostic device 1 according to an embodiment of the present invention. FIG. 2A and FIG. 2B are diagrams illustrating an example of an attachment state of a camera 22 with respect to an ultrasound probe 21. FIG. 2A illustrates an example of the attachment state of the camera 22 with respect to a surface 211 (hereinafter, referred to as a “long-axis surface 211”) along the long-axis direction of the outer peripheral surface of the ultrasound probe 21. FIG. 2B illustrates an example in which the camera 22 is attached to a surface 212 (hereinafter, referred to as a “short-axis surface 212”) along a short-axis direction of the outer circumferential surface of the ultrasound probe 21. FIG. 3 is a block diagram illustrating a main part of a control system of the ultrasound diagnostic device 1.

### Ultrasound Diagnostic Device 1

The ultrasound diagnostic device 1 is used to visualize a shape, a property, or a dynamic state in a subject as an ultrasound image and to perform image diagnosis. As illustrated in FIG. 1, the ultrasound diagnostic device 1 includes an ultrasound diagnostic device body 10, the ultrasound probe 21, and the camera 22. The ultrasound probe 21 and the camera 22 are communicably connected to the ultrasound diagnostic device body 10 via, for example, a cable 23. The ultrasound probe 21 and the camera 22 may be communicably connected to the ultrasound diagnostic device body 10 by wireless communication.

### Ultrasound Probe 21

The ultrasound probe 21 transmits ultrasound waves to a subject, receives ultrasound echoes (reflected ultrasound waves) reflected by the subject, converts the ultrasound echoes into reception signals, and transmits the reception signals to the ultrasound diagnostic device body 10.

As the ultrasound probe 21, an arbitrary electronic scanning probe such as a convex probe, a linear probe, or a sector probe, or a mechanical scanning probe such as a mechanical sector probe may be applied. The ultrasound probe 21 may include a puncture needle guide portion to which a puncture needle is attached and which guides the puncture direction.

The ultrasound probe 21 includes, for example, an acoustic lens, an acoustic matching layer, a transducer array, and a backing material (none of which are illustrated) in this order from the ultrasound wave transmission/reception side (the surface side of the probe head). Note that a protective layer may be arranged on the surface (ultrasound wave transmission/reception surface) of the acoustic lens.

The acoustic lens is a lens that converges ultrasound waves in a slice direction. The slice direction is a direction orthogonal to the scanning direction in which the plurality of transducers are arranged. For example, in a case where a material having a sound velocity lower than that of a living body is used for the acoustic lens, the acoustic lens has a semicylindrical shape in which a central portion in the slice direction is raised. Hereinafter, the slice direction is referred to as a “short-axis direction” and the scan direction is referred to as a “long-axis direction”.

The acoustic matching layer is an intermediate substance for causing an ultrasound wave to efficiently enter the subject, and matches acoustic impedances of the subject and the transducer. The transducer array is formed by, for example, a plurality of strip-shaped transducers arranged in a single row in a scanning direction. The backing material attenuates unnecessary vibration generated in the transducer array.

The ultrasound probe 21 has two long-axis surfaces 211 facing each other in the short-axis direction and two short-axis surfaces 212 facing each other in the long-axis direction. The camera 22 is attached along the long-axis surface 211 or the short-axis surface 212.

A projection (not shown) called an orientation mark is provided on one short-axis surface 212 of the ultrasound probe 21. Generally, in ultrasound diagnostic, how to bring an ultrasound probe into contact with a diagnostic target site is determined with reference to an orientation mark. The orientation mark allows the user to recognize how the ultrasound probe 21 is in contact with the diagnostic target site and to easily understand how the diagnostic target site is rendered in the ultrasound image.

### Camera 22

The camera 22 is attached to the ultrasound probe 21. The camera 22 captures images of the vicinity of the probe scanning position on the body surface of the subject, and transmits an imaging signal to the ultrasound diagnostic device body 10.

The camera 22 is a known imaging device and includes an optical lens, a lens driving device, and an imaging element. It is preferable that the optical lens includes a wide-angle lens having a wider angle of view than the standard lenses. The lens driving device has, for example, an autofocus function of moving the optical lens in an optical axis direction to perform focusing. The imaging element is configured by, for example, a charge-coupled device (CCD) type image sensor, a complementary metal oxide semiconductor (CMOS) type image sensor, or the like. The image sensor captures a subject image formed by the optical lens.

The camera 22 is attached to the ultrasound probe 21 so as to capture an image of the vicinity of the probe scanning position. The camera 22 is removably attached to the outer circumferential surface of the ultrasound probe 21 using an appropriate fixing tool. The manner in which the camera 22 is attached to the ultrasound probe 21 can be changed. The attachment state includes an attachment position and an attachment posture.

The attachment position includes the circumferential position on the outer circumferential surface of the ultrasound probe 21, i.e., the long-axis surface 211 and the short-axis surface 212 of the ultrasound probe 21. The attachment position may include a position in an up-down direction with respect to the ultrasound probe 21 (a distance from a scanning surface).

The attachment posture includes rotation around the optical axis of the camera 22 (roll) and/or rotation around two axes orthogonal to the optical axis (yaw and pitch).

The user can appropriately adjust the attachment state of the camera 22 so that the landmark capable of specifying the probe scanning position is included in the imaging range.

In the present embodiment, it is assumed that the attachment position of the camera 22 is fixed in advance on each of the two long-axis surfaces 211 and the two short-axis surfaces 212 of the ultrasound probe 21, and is appropriately selected from four attachment positions. Further, it is assumed that the attachment posture is that the optical axis of the camera 22 is orthogonal to the scanning surface of the ultrasound probe 21 and the camera 22 can be rotated only around the optical axis. That is, the yaw and the pitch are unchanged with respect to the attachment posture of the camera 22.

Note that the attachment position of the camera 22 may be continuously variable along the outer peripheral surface of the ultrasound probe 21. The attachment posture of the camera 22 may be rotatable around the yaw axis and the pitch axis.

### Ultrasound Diagnostic Device Body 10 (Ultrasound Diagnostic Support Device)

The ultrasound diagnostic device body 10 visualizes a shape, a property, or a dynamic state in the subject as an ultrasound image (a B-mode image) by using a reception signal from the ultrasound probe 21. In the present embodiment, an ultrasound diagnostic support device according to the present invention is applied to the ultrasound diagnostic device body 10.

The ultrasound diagnostic device body 10 includes a system controller 11, a transceiver 12, an ultrasound image generator 13, a camera image generator 14, a display image generator 15, a storage 17, an operation unit 18, and a display unit 19.

The system controller 11 includes a central processing unit (CPU 111) as an arithmetic/control device, a read only memory (ROM 112) and a random access memory (RAM 113) as main storage devices, and the like. A basic program and basic setting data are stored in the ROM 112. Furthermore, an ultrasound diagnostic support program to be executed during diagnosis is stored in the ROM 112. The system controller 11 includes a third hardware processor and a fourth hardware processor.

In the present embodiment, the system controller 11 functions as a camera information setter that sets the camera attachment information. The camera attachment information is information indicating an attachment state of the camera 22 with respect to the ultrasound probe 21. The camera attachment information includes position information indicating the attachment position of the camera 22 and posture information indicating the attachment posture. The system controller 11 functions as a camera information setter, and acquires camera attachment information based on an operation signal from the operation unit 18, for example. The camera information setter is implemented by, for example, the fourth hardware processor.

The system controller 11 also functions as a diagnostic information manager that manages diagnostic information such as an ultrasound image. The system controller 11 functions as a diagnostic information manager, and stores the acquired camera attachment information in the storage 17 in association with the ultrasound image and the camera image. The diagnostic information manager is implemented by the third hardware processor, for example.

The CPU 111 reads a program corresponding to the processing content from the ROM 112, develops the program in the RAM 113, and executes the developed program, thereby centrally controlling the operation of each functional block of the ultrasound diagnostic device body 10. The function of each functional block is implemented by the cooperation of each hardware configuring the functional block and the system controller 11, for example. Note that some or all of the functions of the functional blocks may be implemented by the system controller 11 executing a program.

The transceiver 12 includes a transmission circuit and a reception circuit.

The transmission circuit generates a transmission signal (drive signal) in accordance with an instruction of the system controller 11, and outputs the transmission signal to the ultrasound probe 21. The transmission circuit includes, for example, a clock generation circuit, a delay circuit, a pulse generation circuit, and a pulse width setter.

The reception circuit receives a reception signal from the ultrasound probe 21 in accordance with an instruction from the system controller 11, and outputs the reception signal to the ultrasound image generator 13. The reception circuit includes, for example, an amplifier, an A/D conversion circuit, and a phasing addition circuit.

The ultrasound image generator 13 generates an ultrasound image indicating the internal state of the subject based on a reception signal obtained by transmission and reception of ultrasound waves in accordance with an instruction from the system controller 11. The ultrasound image is, for example, a B-mode tomographic image, a blood flow spectrum by Doppler, or a blood flow distribution image. The data of the generated ultrasound image is stored in, for example, the storage 17. The ultrasound image generator 13 is implemented by, for example, a first hardware processor.

The camera image generator 14 generates a camera image based on an imaging signal from the imaging element of the camera 22 in accordance with an instruction from the system controller 11. The vicinity of the probe scanning position is rendered in the camera image. The user can specify the probe scanning position based on the camera image. The data of the generated camera image is stored in, for example, the storage 17 in association with the ultrasound image. The camera image generator 14 is implemented by, for example, a second hardware processor.

The display image generator 15 generates an image for display on the basis of the image data from the ultrasound image generator 13 and the camera image generator 14 according to an instruction of the system controller 11. The display image includes, for example, an ultrasound image and a camera image. The display image generator 15 includes a digital scan converter (DSC) and the like that performs coordinate conversion and pixel interpolation according to the type of the ultrasound probe 21. The generated data of the image for display is output to the display unit 19.

The ultrasound image generator 13, the camera image generator 14, and the display image generator 15 are configured by dedicated or general-purpose hardware (electronic circuit) corresponding to each process, such as a digital signal processor (DSP), an application specific integrated circuit (ASIC), or a programmable logic device (PLD), and implement each function in cooperation with the system controller 11.

The operation unit 18 receives, for example, an input of a command instructing to start diagnosis or the like or information on a subject. The operation unit 18 includes, for example, an operation panel having a plurality of input switches, a keyboard, and a trackball. Note that the operation unit 18 may be formed of a touch panel provided integrally with the display unit 19.

The user can instruct the execution of the ultrasound diagnostic process by using the operation unit 18. In addition, the user can set the camera attachment information using the operation unit 18. A specific method of setting the camera attachment information will be described later.

The display unit 19 includes, for example, a liquid crystal display, an organic EL display, a CRT display, or the like. The display unit 19 displays the ultrasound image and the camera image based on the data for display from the display image generating part 15 according to the instruction of the system controller 11.

Although illustration is omitted, the ultrasound diagnostic device body 10 may include a communicator that transmits and receives various information to and from an external device (e.g., a personal computer or a cloud storage on the Internet) connected to a communication network such as a wired/wireless local area network (LAN). The communicator includes, for example, various interfaces such as a network interface card (NIC), a modulator-demodulator (MODEM), and a universal serial bus (USB). In addition, a communication interface for short-range wireless communication such as near field communication (NFC) or Bluetooth® can be applied to the communication unit.

### Diagnostic Screen

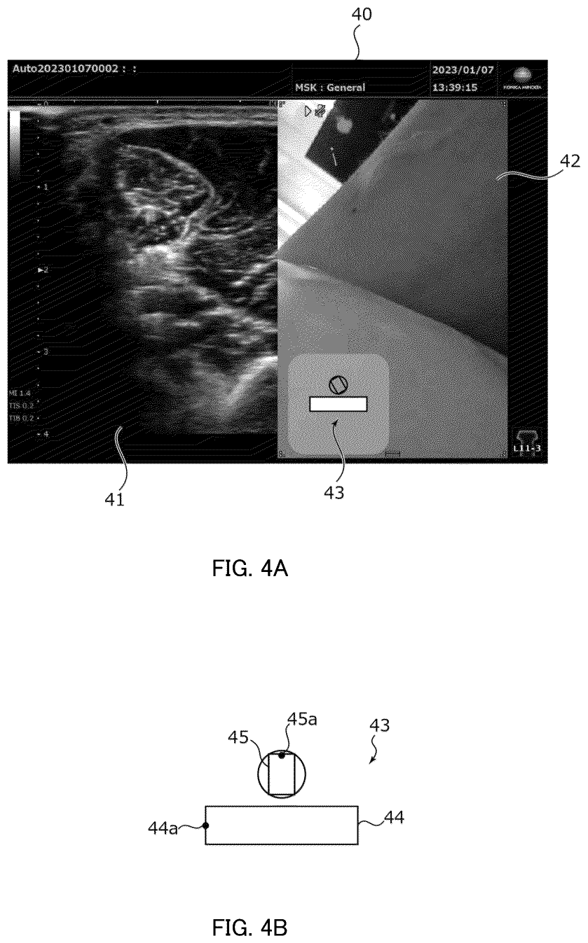

FIGS. 4A and 4B are diagrams illustrating an example of a diagnostic screen 40 displayed at the time of diagnosis. FIG. 4A illustrates the entire diagnostic screen 40, and FIG. 4B illustrates extracted camera attachment information 43 included in the diagnostic screen 40.

As illustrated in FIG. 4A, the diagnostic screen 40 includes an ultrasound image 41, a camera image 42, and camera attachment information 43. The probe scanning position is depicted in the camera image 42. In addition, in the camera attachment information 43, how the camera 22 is attached to the ultrasound probe 21 is indicated by an icon.

As illustrated in FIG. 4B, the icon indicating the camera attachment information 43 includes a probe icon 44 indicating the ultrasound probe 21 and a camera icon 45 indicating the camera 22. Note that the probe icon 44 and the camera 22 illustrated in FIG. 4B are an example of the case where the camera attachment information 43 is displayed by an icon. A drawing form (e.g., a figure, a color, or the like) of the icon is not limited to that illustrated in FIG. 4B as long as the attachment state of the camera 22 with respect to the ultrasound probe 21 can be visually recognized.

Note that the camera attachment information 43 may be displayed as text information together with the icon or instead of the icon.

The probe icon 44 is arranged at a fixed position in a display area of the camera attachment information 43. The probe icon 44 has a form that reminds of a scanning surface of the ultrasound probe 21. In FIG. 4B, the probe icon 44 is drawn with a rectangular frame that can distinguish between the long-axis surface 211 and the short-axis surface 212 of the ultrasound probe 21. The long side of the probe icon 44 corresponds to the long-axis surface 211 of the ultrasound probe 21. The short side of the probe icon 44 corresponds to the short-axis surface 212 of the ultrasound probe 21.

The probe icon 44 has a probe reference mark 44a indicating a reference plane of the outer circumferential surface (the long-axis surface 211 and the short-axis surface 212) of the ultrasound probe 21. The probe reference mark 44a is placed, for example, corresponding to the surface (here, the one short-axis surface 212) on which the orientation mark is provided.

The camera icon 45 is arranged adjacent to the long side or the short side of the probe icon 44. In FIG. 4B, the camera icon 45 is drawn with a rectangular frame representing an image-capturing region. The camera icon 45 has a camera reference mark 1a indicating a reference for the attachment posture of the camera 22. The camera reference mark 45a is arranged, for example, at a position indicating an upper side in the camera image 42 of the diagnostic screen 40.

The camera icon 45 is arranged around the probe icon 44 based on camera attachment information preset by the camera information setter (system controller 11). The user can know the attachment position of the camera 22 with respect to the ultrasound probe 21 from the position of the camera icon 45 with respect to the probe icon 44. Further, the user can know the attachment posture of the camera 22, that is, the rotation around the optical axis, from the rotation state of the camera icon 45.

For example, in the case of the camera attachment information 43 illustrated in FIG. 4B, the user can know that the camera 22 is arranged on one long-axis surface 211 of the ultrasound probe 21. The user can also know that the up-down direction of the camera image 42 corresponds to the short-axis direction of the ultrasound probe 21 and the upper side of the camera image 42 is the distal side with respect to the ultrasound probe 21.

As illustrated in FIG. 4A, the ultrasound image 41, the camera image 42, and the camera attachment information 43 are displayed on the same diagnostic screen 40. With the diagnostic screen 40, the user can understand how the camera image 42 is captured by the camera 22 attached to the ultrasound probe 21, and can easily and accurately grasp the probe scanning position with respect to the subject. Thus, the objectivity of diagnosis based on the ultrasound image 41 is improved. In addition, since the probe scanning position can be easily reproduced with reference to the camera image 42 and the camera attachment information 43, the reproducibility of ultrasound diagnostic is improved.

### Setting of Camera Attachment Information

In the present embodiment, the camera attachment information is set in advance before the start of ultrasound diagnostic. The camera attachment information may be manually set by the user or automatically set, for example. The camera attachment information is set on the basis of an actual attachment state of the camera 22 with respect to the ultrasound probe 21.

### Setting Example 1 of Camera Attachment Information

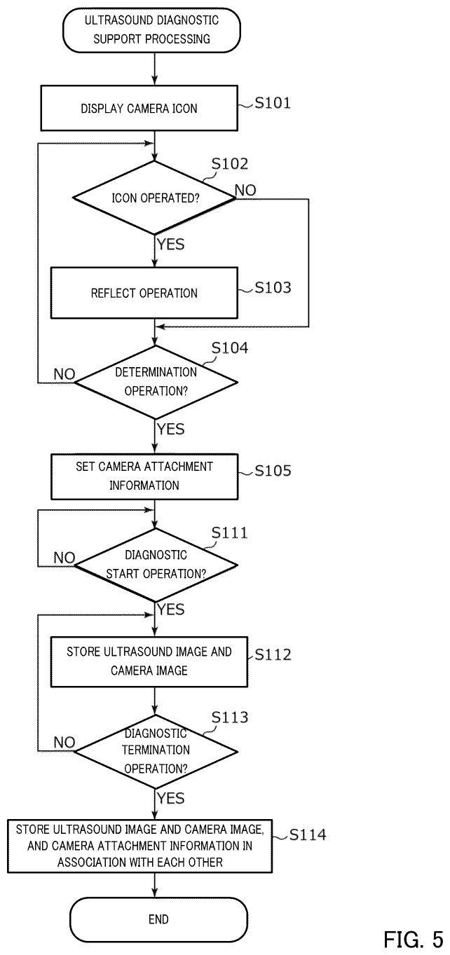

FIG. 5 is a flowchart illustrating an example of ultrasound diagnostic support processing executed in a case where camera attachment information is manually set. This processing is implemented by the CPU 111 of the system controller 11 executing the ultrasound diagnostic support program stored in the ROM 112 with the start of the diagnostic mode in the ultrasound diagnostic device 1, for example.

In the setting example 1, the system controller 11 presents an icon indicating the attachment state of the camera 22 with respect to the ultrasound probe 21, and sets the camera attachment information based on an icon operation using the icon.

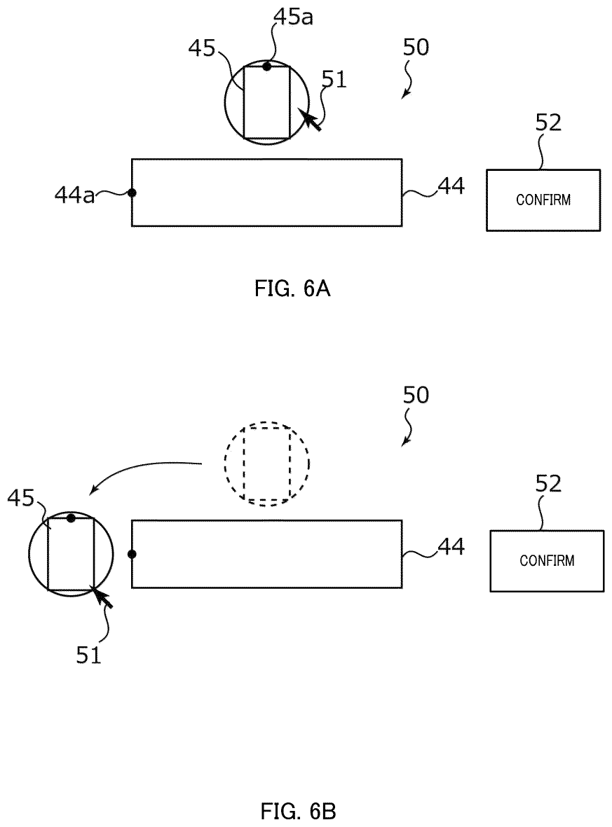

In particular, in step S101, the system controller 11 controls the display unit 19 to display the setting screen 50 (processing as a camera information setter). As illustrated in FIGS. 6A and 6B, the probe icon 44 and the camera icon 45 are displayed on the setting screen 50. The probe icon 44 and the camera icon 45 are the same as those constituting the camera attachment information 43 displayed on the diagnostic screen 40.

For example, the user changes the position of the camera icon 45 with respect to the probe icon 44 and the rotation state of the camera icon 45 according to the actual attachment state of the camera 22 by using a drawing tool 51 such as a cursor displayed on the setting screen 50. The user can intuitively recognize the attachment state of the camera 22 by the icon. Further, the user can confirm the camera attachment information by a confirmation operation of clicking the confirmation button 52 displayed on the setting screen 50.

In step S102, the system controller 11 determines, based on an operation signal from the operation unit 18, whether or not an icon operation has been performed by the user (processing as a camera information setter). When the icon operation is performed (“YES” in step S102), the process proceeds to step S103. When the icon operation is not performed (“NO” in step S102), the process proceeds to step S104.

In step S103, the system controller 11 acquires the content of the operation performed using the drawing tool 51, and reflects the content in the display form of the camera icon 45 (processing as a camera information setter). For example, in the setting screen 50 illustrated in FIG. 6A, when the drawing tool 51 is moved to a position along the short side of the probe icon 44 with being aligned with the camera icon 45, the screen changes to the setting screen 50 illustrated in FIG. 6B.

In step S104, the system controller 11 determines, based on an operation signal from the operation unit 18, whether or not a confirmation operation has been performed by the user (processing as a camera information setter). When the confirmation operation is performed (“YES” in step S104), the process proceeds to step S105. If the confirmation operation has not been performed (“NO” in step S104), the process proceeds to step S102 and the processes in steps S102 to S104 are repeated.

In step S105, the system controller 11 sets the camera attachment information on the basis of the position coordinates and the rotation state of the camera icon 45 determined on the setting screen 50 (processing as a camera information setter). The set camera attachment information is temporarily stored in the RAM 113, for example.

Next, in step S111, the system controller 11 determines, based on an operation signal from the operation unit 18, whether or not a diagnostic start operation has been performed. The diagnosis start operation is, for example, an operation in which the user presses a diagnosis start button of the operation unit 18. If the diagnostic start operation has been performed (“YES” in step S111), the process proceeds to step S112.

In step S112, the system controller 11 stores the ultrasound image and the camera image in association with each other. The data of the ultrasound image and the camera image are, for example, temporarily stored in the RAM 113. The ultrasound image and the camera image may be still images or moving images.

In step S113, the system controller 11 determines whether a diagnostic termination operation has been performed. The diagnostic termination operation is, for example, an operation of pressing a diagnostic end button of the operation unit 18 by the user. When the diagnosis end operation is performed (“YES” in step S113), the process proceeds to step S114. If the diagnostic ending operation has not been performed (NO in step S113), the process proceeds to step S112 and repeats the processing in steps S112 and S113.

In step S114, the system controller 11 stores the ultrasound image and the camera image stored in step S112 and the camera attachment information set in step S105 in association with each other in the storage unit 17, for example. The stored ultrasound image, camera image, and camera attachment information are, for example, read out at the time of diagnosis and displayed on the diagnostic screen 40 (see FIG. 4A).

### Setting Example 2 of Camera Attachment Information

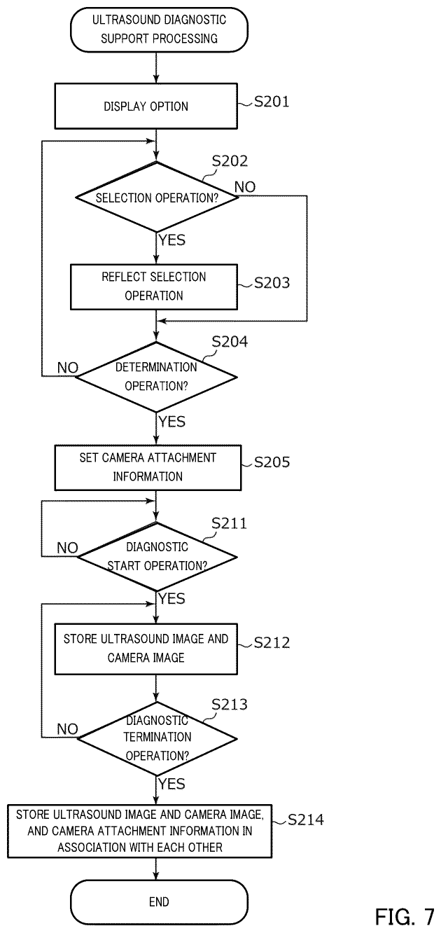

FIG. 7 is a flowchart illustrating another example of the ultrasound diagnostic support processing executed in a case where the camera attachment information is manually set. A main difference from the flowchart illustrated in FIG. 5 is processing as the camera information setter in steps S201 to S205.

In the setting example 2, a plurality of options indicating the attachment state of the camera 22 with respect to the ultrasound probe 21 are presented, and the camera attachment information is set based on an operation of selecting one of the plurality of options.

In particular, in step S201, the system controller 11 controls the display unit 19 to display the setting screen 60 (processing as a camera information setter). As illustrated in FIGS. 7A and 7B, four candidate position buttons 61 to 64 serving as candidates for the attachment position of the camera 22 are arranged around the probe icon 44 on the setting screen 60.

The user can visually recognize the option indicating the attachment state of the camera 22 with respect to the ultrasound probe 21 by the candidate position button 61. The user selects the attachment position of the camera 22 from among the candidate position buttons 61 to 64 displayed on the setting screen 60, for example.

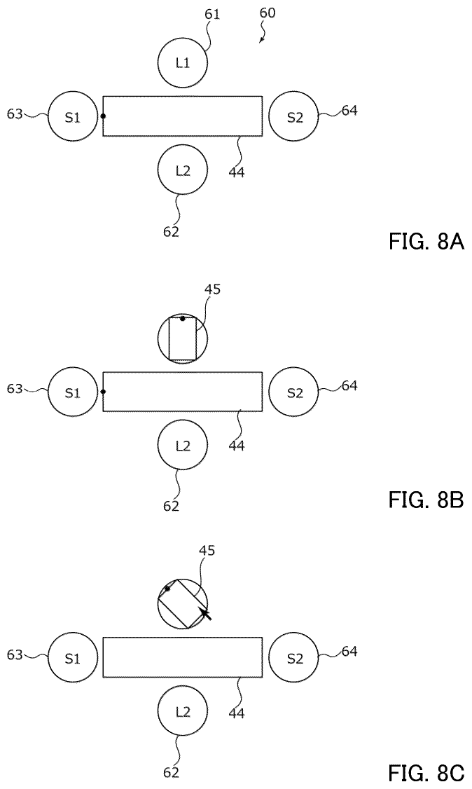

In step S202, the system controller 11 determines, based on an operation signal from the operation unit 18, whether or not a selection operation has been performed by the user (processing as a camera information setter). When the selection operation has been performed (“YES” in step S202), the process proceeds to step S203. When the selection operation has not been performed (“NO” in step S202), the process proceeds to step S204. In step S203, the system controller 11 reflects the selection operation in the setting screen 60 (processing as a camera information setter). For example, in the setting screen 60 illustrated in FIG. 8A, when a selection operation of a candidate position button 61 is performed, the setting screen 60 illustrated in FIG. 8B is set. In FIG. 8B, the candidate position button 61 changes to the camera icon 45, indicating that the candidate position L1 has been selected as the attachment position of the camera 22.

In step S204, the system controller 11 determines, based on an operation signal from the operation unit 18, whether or not a confirmation operation has been performed by the user (processing as a camera information setter). When the confirmation operation has been performed (“YES” in step S204), the process proceeds to step S205. If the confirmation operation has not been performed (“NO” in step S204), the process proceeds to step S202 and the processes in steps S202 to S204 are repeated.

In step S205, the system controller 11 sets the camera attachment information on the basis of the candidate positions determined on the setting screen 60 (processing as a camera information setter). The set camera attachment information is temporarily stored in the RAM 113, for example.

The processing as the diagnostic information manager of steps S211 to S214 is the same as steps S111 to S114 of FIG. 5, and thus the description will be omitted.

Note that in the present setting example 2, after the camera icon 45 is displayed, as in the setting example 1, the rotation state of the camera icon 45 may be allowed to be changed by an icon operation (see FIG. 8C). Furthermore, in the setting example 2, the options indicating the attachment positions of the cameras 22 may be presented by text information instead of the candidate position buttons 61 to 64.

### Setting Example 3 of Camera Attachment Information

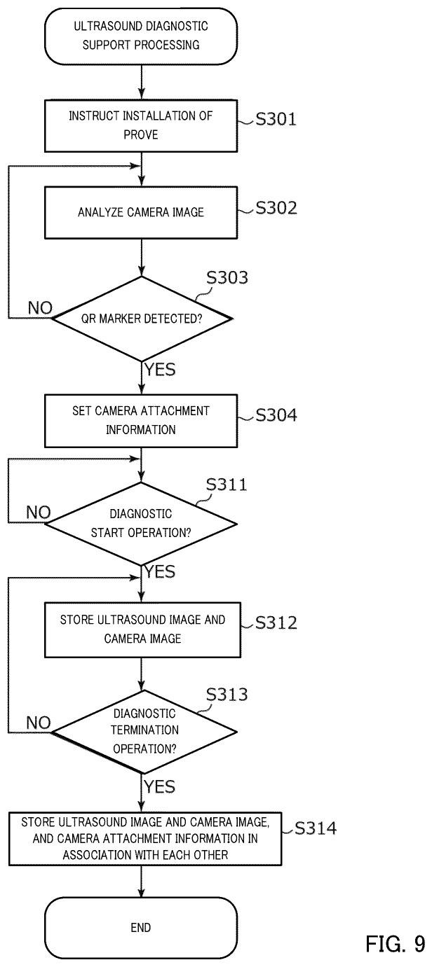

FIG. 9 is a flowchart illustrating an example of the ultrasound diagnostic support processing that is executed when the camera attachment information is automatically set. A main difference from the flowchart illustrated in FIG. 5 is processing as the camera information setter of steps S301 to S304.

In the setting example 3, the system controller 11 acquires a camera image captured by the camera 22 in a state where the ultrasound probe 21 is installed at a predetermined position of a calibration sheet having identification information capable of identifying the attachment state of the camera 22 with respect to the ultrasound probe 21, and sets the camera attachment information on the basis of the identification information included in the camera image.

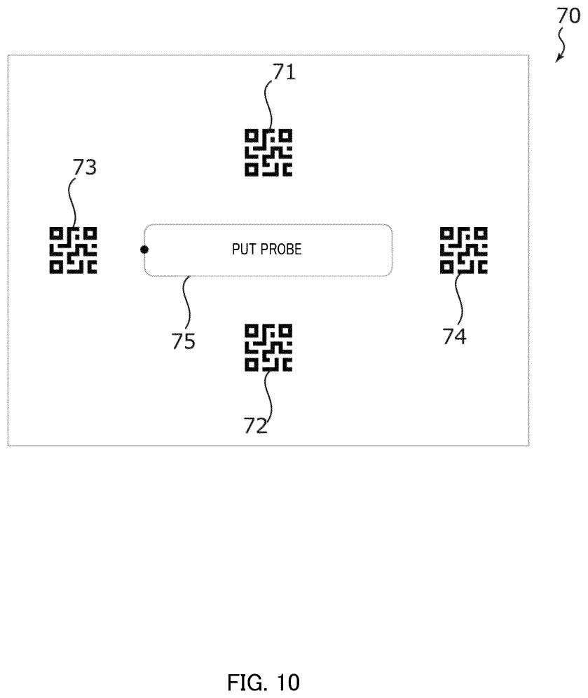

Specifically, a calibration sheet 70 illustrated in FIG. 10 is used. The calibration sheet 70 has a probe installation frame 75 indicating the installation position of the ultrasound probe 22. Around the probe installation frame 75, different pieces of identification information 71 to 74 (e.g., QR markers) are arranged at four places that are candidates for the attachment position of the camera 22. Information indicating the attachment position of the camera 22 is associated with the identification information 71 to 74.

In step S301, the system controller 11 controls the display unit 19 to display an instruction to the user to place the ultrasound probe 21 on the calibration sheet 70. The user installs the ultrasound probe 21 in accordance with the probe installation frame 75 of the calibration sheet 70 in accordance with the displayed instruction.

In step S302, the system controller 11 analyzes the camera image acquired by the camera 22 (processing as a camera information setter).

In step S303, the system controller 11 determines, based on the analysis result of the camera image, whether or not identification information has been detected (processing as a camera information setter). When the identification information is detected (“YES” in step S303), the process proceeds to step S304. If the identification information is not detected (“NO” in step S303), the process proceeds to step S302 and repeats the processing in steps S302 and S303.

In step S304, the system controller 11 sets the camera attachment information on the basis of the detected identification information (processing as a camera information setter). The set camera attachment information is temporarily stored in the RAM 113, for example.

Since processing in steps S311 to S314 as the diagnostic information manager is the same as steps S111 to S114 in FIG. 5, description thereof will be omitted.

Note that in the present setting example 3, after the attachment position of the camera 22 is determined based on the detection results of the identification information 71 to 74, the camera icon 45 may be displayed as in the setting example 1 such that the rotation state of the camera icon 45 can be changed by an icon operation.

As described above, the ultrasound diagnostic device body 10 according to the embodiment includes the following features alone or in appropriate combination.

That is, the ultrasound diagnostic device body 10 (ultrasound support device) of the present embodiment includes the ultrasound image generator 13 that generates an ultrasound image based on an ultrasound signal from the ultrasound probe 21 transmitting and receiving ultrasound waves, the camera image generator 14 that generates a camera image based on an imaging signal from the camera 22 attached to the ultrasound probe 21, and the diagnostic information manager that stores camera attachment information indicating an attachment state of the camera 22 with respect to the ultrasound probe 21 in association with the ultrasound image and the camera image. The diagnostic information manager is implemented by the system controller 11 of the ultrasound diagnostic device body 10.

An ultrasound diagnostic support method according to an embodiment generates an ultrasound image on the basis of an ultrasound signal from the ultrasound probe 21 that transmits and receives ultrasound waves, generates a camera image on the basis of an imaging signal from a camera attached to the ultrasound probe 21, and stores camera attachment information indicating an attachment state of the camera with respect to the ultrasound probe in association with the ultrasound image and the camera image (steps S111 to S114 in FIG. 5).

In the embodiment, the system controller 11 executes the ultrasound diagnostic support program to implement the ultrasound diagnostic support device according to the present invention. That is, the ultrasound diagnostic support program causes the system controller 11 (computer) to perform processing for generating an ultrasound image based on ultrasound signals from the ultrasound probe 21 that transmits and receives ultrasound waves, processing for generating a camera image based on imaging signals from the camera 22 attached to the ultrasound probe 21, and processing for storing camera attachment information indicating the attachment state of the camera 22 with respect to the ultrasound probe 21 in association with the ultrasound image and the camera image (steps S111 to S114 in FIG. 5).

The ultrasound diagnostic support program can be provided, for example, via a computer-readable portable storage medium (including, for example, an optical disc, a magneto-optical disk, and a memory card) in which the program is stored. Furthermore, for example, the ultrasound diagnostic support program can be provided by being downloaded from a server that holds the program via a network.

Furthermore, the ultrasound diagnostic device 1 according to the embodiment includes the ultrasound diagnostic device body 10 to which the ultrasound diagnostic support device is applied, the ultrasound probe 21, and the camera 22.

According to the embodiment, since it is possible to grasp how the camera 22 is attached to the ultrasound probe 21 from the camera attachment information, it is possible to grasp whether the camera image is an image that is obtained in a state where the ultrasound probe 21 is in contact with the subject. Therefore, since the probe scanning position when the ultrasound image is acquired can be easily specified from the camera image, the objectivity of the diagnostic based on the ultrasound image is improved. In addition, since the probe scanning position can be easily reproduced with reference to the camera image and the camera attachment information, the reproducibility of ultrasound diagnostic is improved. Note that the attachment position and the attachment posture of the camera 22 with respect to the ultrasound probe 21 are preferably adjusted such that landmarks capable of identifying the probe scanning position are drawn in the camera image.

In the ultrasound diagnostic device body 10 (ultrasound diagnostic support device), the camera attachment information includes position information indicating the attachment position of the camera 22 with respect to the ultrasound probe 21 and posture information indicating the attachment posture of the camera 22. Since the user can more accurately grasp how the camera 22 is attached to the ultrasound probe 21, the accuracy of the probe scanning position specified from the camera image is improved.

In the ultrasound diagnostic device body 10 (ultrasound diagnostic support device), the system controller 11 as a diagnostic information management part displays an ultrasound image, a camera image, and camera attachment information associated with each other on the same screen. More specifically, the system controller 11 (diagnostic information manager) displays the camera attachment information in a superimposed manner on the camera image. The user can easily grasp the relationship between the ultrasound image and the probe scanning position on the same screen.

Furthermore, the ultrasound diagnostic device body 10 (ultrasound diagnostic support device) includes a camera information setter that can set camera information. The camera information setter is implemented by the system controller 11 of the ultrasound diagnostic device body 10. The ultrasound diagnostic device body 10 can easily acquire the attachment state of the camera 22 with respect to the ultrasound probe 21.

In the ultrasound diagnostic device body 10 (ultrasound diagnostic support device), the system controller 11 as a camera information setting part presents an icon indicating the attachment state of the camera 22 with respect to the ultrasound probe 21, and sets the camera attachment information based on an icon operation using the icon. The user can easily set the attachment state of the camera 22 by an intuitive operation using the icon.

In the ultrasound diagnostic device body 10 (ultrasound diagnostic support device), the system controller 11 as a camera information setting part presents a plurality of options indicating the attachment state of the camera 22 with respect to the ultrasound probe 21, and sets the camera attachment information on the basis of a selection operation from the plurality of options. The user can easily set the attachment state of the camera 22 by the selection operation from the plurality of options.

In the ultrasound diagnostic device body 10 (ultrasound diagnostic support device), the system controller 11 as a camera information setter acquires a camera image captured by the camera 22 in a state where the ultrasound probe 21 is installed at a predetermined position of a calibration sheet having identification information capable of identifying an attachment state of the camera 22 with respect to the ultrasound probe 21, and sets the camera attachment information on the basis of the identification information included in the camera image. The user can easily set the attachment state of the camera 22 only by installing the ultrasound probe 21 at a predetermined position on the calibration sheet.

In the ultrasound diagnostic device 1, the attachment position and/or attachment posture of the camera 22 with respect to the ultrasound probe 21 is changeable. Furthermore, the camera 22 is attach to and detachable from the ultrasound probe 21. The camera 22 preferably has a wide-angle lens. The user can easily adjust the attachment position and the attachment posture of the camera 22 with respect to the ultrasound probe 21 so that the landmark capable of specifying the probe scanning position is drawn in the camera image.

Although the invention made by the present inventors has been specifically described above based on the embodiments, the present invention is not limited to the above-described embodiments, and modifications can be made without departing from the spirit and scope of the invention.

In the embodiment, the ultrasound diagnostic support device of the present invention is applied to the ultrasound diagnostic device body 10, but a part or all of the functions as the ultrasound diagnostic support device may be incorporated in the ultrasound probe 21.

Further, the attachment position of the camera 22 may be continuously variable in each of the two long-axis surfaces 211 and the two short-axis surfaces 212 of the ultrasound probe 21. Furthermore, the attachment posture of the camera 22 may be variable in yaw and pitch. In this case, the camera attachment information is rendered in a three dimensional display.

Furthermore, a detection device such as an acceleration sensor or a gyro sensor may be built into the ultrasound probe 21 and/or the camera 22 such that the attachment state of the camera 22 with respect to the ultrasound probe 21 can be automatically acquired.

Although embodiments of the present invention have been described and illustrated in detail, the disclosed embodiments are made for purpose of illustration and example only and not limitation. The scope of the present invention should be interpreted by terms of the appended claims.

What is claimed is:

1. An ultrasound diagnostic support device, comprising:
a first hardware processor that generates an ultrasound image based on an ultrasound signal from an ultrasound probe that transmits and receives ultrasound waves;
a second hardware processor that generates a camera image based on an imaging signal from a camera attached to the ultrasound probe; and
a third hardware processor that stores camera attachment information indicating an attachment state of the camera with respect to the ultrasound probe in association with the ultrasound image and the camera image.

2. The ultrasound diagnostic support device according to claim 1, wherein the camera attachment information includes posture information indicating an attachment posture of the camera and position information indicating an attachment position of the camera with respect to the ultrasound probe.

3. The ultrasound diagnostic support device according to claim 1, wherein the third hardware processor displays on the same screen the ultrasound image, the camera image, and the camera attachment information associated with each other.

4. The ultrasound diagnostic support device according to claim 3, wherein the third hardware processor displays the camera attachment information in a superimposed manner on the camera image.

5. The ultrasound diagnostic support device according to claim 1, wherein a fourth hardware processor that sets the camera attachment information.

6. The ultrasound diagnostic support device according to claim 5, wherein the fourth hardware processor presents an icon indicating the attachment state of the camera with respect to the ultrasound probe, and sets the camera attachment information based on an icon operation using the icon.

7. The ultrasound diagnostic support device according to claim 5, wherein the fourth hardware processor presents a plurality of options indicating the attachment state of the camera with respect to the ultrasound probe, and sets the camera attachment information based on an operation of selecting one of the plurality of options.

8. The ultrasound diagnostic support device according to claim 5,
wherein the fourth hardware processor acquires the camera image captured by the camera in a state where the ultrasound probe is installed at a predetermined position of a calibration sheet including identification information for identifying an attachment state of the camera with respect to the ultrasound probe, and
wherein the fourth hardware processor sets the camera attachment information based on the identification information included in the camera image.

9. An ultrasound diagnostic device comprising:
an ultrasound diagnostic device body to which the ultrasound diagnostic support device according to claim 1 is applied; and
the ultrasound probe; and
the camera.

10. The ultrasound diagnostic device according to claim 9, wherein the camera changes an attachment posture and/or an attachment position with respect to the ultrasound probe.

11. The ultrasound diagnostic device according to claim 9, wherein the camera is attachable to and detachable from the ultrasound probe.

12. The ultrasound diagnostic device according to claim 9, wherein the camera includes a wide-angle lens.

13. An ultrasound diagnostic support method comprising:
generating an ultrasound image based on an ultrasound signal from an ultrasound probe that transmits and receives ultrasound waves;
generating a camera image based on an imaging signal from a camera attached to the ultrasound probe; and
storing camera attachment information indicating an attachment state of the camera with respect to the ultrasound probe in association with the ultrasound image and the camera image.

14. A computer-readable non-transitory recording medium storing a program for causing a computer to execute predetermined processing, the predetermined processing comprising:
processing of generating an ultrasound image based on an ultrasound signal from an ultrasound probe that transmits and receives ultrasound waves;
processing of generating a camera image based on an imaging signal from a camera attached to the ultrasound probe; and
processing of storing camera attachment information indicating an attachment state of the camera with respect to the ultrasound probe in association with the ultrasound image and the camera image.
